(19) United States
(12) Patent Application Publication
(10) Pub. No.: US 2008/0114232 A1
(43) Pub. Date: May 15, 2008

(54) Ergometry Belt

(75) Inventor: Amos Gazit, Tel Aviv (IL)

Correspondence Address: LOWE HAUPTMAN HAM & BERNER, LLP, 1700 DIAGONAL ROAD, SUITE 300, ALEXANDRIA, VA, 22314, US

(73) Assignee: TAPUZ MEDICAL TECHNOLOGIES (T.M.T. 2004) LTD., Petach Tikva (IL)

(21) Appl. No.: 11/719,338

(22) PCT Filed: Nov. 13, 2005
(86) PCT No.: PCT/IL05/01190

§ 371 Date: Jul. 18, 2007

(30) Foreign Application Priority Data
Nov. 16, 2004 (IL) 165232

(51) Int. Cl. A61B 5/04

(52) U.S. Cl. 600/390

(57) ABSTRACT

An ergometry belt particularly for use in ergometry (ECG stress test) is disclosed. The ergometry belt—according to the present invention—made of a flexible and stretchable material, a plurality of electrodes disposed on the ergometry belt so that stretching of the belt will locate the electrodes in predetermined locations, a plurality of conductors adapted to be connected to a respective electrode, and a cable connected at one end thereof to each conductor and at another end thereof adapted to be connected to an ECG measuring device.

### FIELD OF THE INVENTION

The present invention relates to the field of accessories used in devices for medical testing. More specifically, the present invention relates to an electrodes belt for use in electrocardiogram (ECG) stress test (hereinafter called Ergometry)

### BACKGROUND OF THE INVENTION

The regular (rest) ECG test is a test, which is performed by means of an appropriate testing device. The device measures the potential differences between various selected points on the body of a person to be examined (hereinafter called “patient”). These potential differences mirror the electronic activities of the heart muscle. The measurement is done by way of attaching electrodes to the body of a patient. These electrodes then transfer the electronic information to the testing device. For this measurement 10 electrodes are attached to the skin at specific anatomical locations: two electrodes are attached to the patients arms, two are attached to the patient's legs and 6 electrodes are attached around the chest wall in points called V1 to V6.

The ECG Stress test is a very common test used to detect Ischemic Heart Disease. In the test, the examined patient has to make a physical effort like running on a treadmill or riding a bicycle, while his pulse, blood pressure and ECG are monitored continuously. The load of the patient work is elevated gradually according to a protocol (several protocols are commonly used) until the patient reaches the target pulse level (that is calculated for every patient based on age, sex and physical condition). The physician is looking for ECG changes during the test, suggesting that the heart suffers lack of Oxygen at a certain stress level. If the patient develops chest pain or ECG changes the test is usually stopped. A cardiac stress test can detect a hidden heart disease at about 70% sensitivity level. The test is widely used as a screening test for heart disease in certain high-risk populations, as well as a routine follow up tests for known heart patient.

Since the patient has to move during ECG recording at a stress test, placement of the electrodes becomes a major problem. The ECG waveform is very sensitive to movement. If electrodes contacts are not secured, the recording becomes too noisy for any diagnosis. In order to secure the electrodes on the skin, male patients chest has to be shaved. Ten sticking disposable ECG electrodes are usually attached to the skin (Lately a new expensive system have also been used consisting on active vacuum electrodes). An ECG lead wire is connecting each electrode to a main cable that is connected to the ECG machine. The lead wires and cable also have to be secured on the body, in order to allow freedom of movement to the patient, as well as ECG recording noise due to wires movement. Preparation of a patient for the test usually takes more time than the test itself. It has to be done by a trained technician. Since a cardiologist is needed too, the test becomes quite expensive and hard to get. The result is that in many places in the world the test is not performed enough, leading to unnecessary mortality and morbidity of heart patient who could be saved by earlier diagnosis of their disease.

In order to ease the performance of rest ECG tests, an apron that includes electrodes was developed. This apron is described in details in U.S. Pat. No. 6,205,346 and U.S. Pat. No. 6,341,229 to Sharon Akiva. This apron is made for tests when the patient is resting and does not suitable for ergometry ECG test, which is performed in stress. For ergometry ECG tests the electrodes are located in a different location on the patient's body and the electrodes must be tightened to the patient's body, since the patient is in movement during the test.

The present invention is an electrodes belt specifically designed for Ergometry. The belt is easily placed on the patient's during the stress test, and provides contact for the necessary electrodes at the correct anatomical location.

### SUMMARY OF THE INVENTION

The present invention relates to an electrodes belt for ergometry. The belt of the present invention is comprised of a belt cast from flexible material to be tied around the chest of the patient, 9 electrodes inlaid within the belt in predetermined places. Either curved or spiraled conductors are cast within flexible material and each conductor is connected to an electrode on one side and a patient cable from the other side. The patient cable has a connector for the ECG device.

The belt can be stretch to fit people of different sizes, man and women alike. Just by placing the belt at the center of the chest, and than stretching the belt to fit the body and closing the buckle behind the back, the belt fit to the body size and the electrodes are proportionally spread apart to fit the right location.

The ergometry belt of the present invention has several advantages:

The electrodes are attached to the belt by means of bridges or membrane of flexible material. This method enables shock observing during movement of the patient, while the bridges or membrane serve as a buffer that reduce the movement that is transferred to the electrode, thus improving ECG signal quality.

Each electrode is bulging towards the body, so when the belt is stretched, the bridges or membrane create a power vector that pushes the electrode towards the body, thus creating positive pressure of the electrode on the skin, avoid monuments and enable better contact between the electrodes and the body. Because of this positive pressure, shaving of male patients is unnecessary.

Since all the conductor pass within the belt, there are only one cables (instead of 10) connecting the belt to the ECG device. It is much more easy to conduct the test, without tangling wires that interfere with the patient's movement and also causing ECG noise.

It is possible to embed snap connectors instead of electrodes on the belt. Then a disposable stretchable thin layer is places between the belt and the body. On this layer disposable ECG electrodes are embedded. The electrodes are connected to the snap connectors, and when the belt is stretched to fit the body the electrodes move with the belt since the disposable layer is also stretchable. This disposable layer of electrodes is replaced at every test, and so every patient's body is touching a clean new surface.

According to the teachings of the present invention there is provided an ergometry belt particularly for use in ergometry ECG tests is provided, comprising this ergometry belt made of a flexible and stretchable material, a plurality of electrodes disposed on the ergometry belt so that stretching of the apron will locate the electrodes in predetermined locations, a plurality of conductors adapted to be connected to a respective electrode, and a cable connected at one end thereof to each conductor and at another end thereof adapted to be connected to an ECG measuring device.

According to further features in the described preferred embodiment the ergometry belt is provided, wherein the conductors are located within the ergometry belt material. And only the patient cable connects the belt to the ECG device.

In another preferred embodiment, the ergometry belt is provided, wherein the electrodes are placed on the patient's body at the correct anatomical places that are used to perform a ECG stress test, when the ergometry belt is worn.

In another preferred embodiment, the ergometry belt is provided, wherein each of the electrodes is assembled to the ergometry belt by means of a number of flexible strips that are pushed out by the electrode when the ergometry belt is worn and the flexible strips react to push back the electrode to the patient's body in order to tighten it.

According to further features and for more hygienic test, the ergometry belt is provided, wherein instead of the plurality of electrodes a plurality of snaps, to which electrodes can be snapped, are disposed on the ergometry belt and wherein the ergometry belt further includes a disposal flexible thin layer with a plurality of electrodes that are disposed on the thin layer in accordance with the ergometry belt snaps' location. The flexible thin layer is attached to the ergometry belt in order to snap the electrodes to the ergometry belt in order to wear it with the attached flexible thin layer for performing an ergometry test

In yet another preferred embodiment the ergometry belt is provided, wherein a flexible membrane is used instead or added to the number of flexible strips.

### BRIEF DESCRIPTION OF THE FIGURES

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the figures:

FIG. 1 illustrates the ergometry belt.

FIG. 2 illustrates the connection of an electrode to the belt.

FIG. 3 illustrates a cross section of the electrode's connection area and the way of the electrode tightening when the belt is worn.

### DESCRIPTION OF THE PREFERRED EMBODIMENTS

The present invention is an ergometry belt to be worn on a patient's body for stress ECG testing.

The ergometry belt, according to the present invention, is made of:

A belt made of flexible and stretchable material, to be worn on the patient's body.

A plurality of electrodes that are disposed on the belt, so the belt when it worn—attaches 10 electrodes to the surface of the patient's body at the correct anatomical places used to perform a cardiac stress test.

A plurality of conductors adapted to be connected to a respective electrode.

A cable connected at one end thereof to each conductor and at another end thereof adapted to be connected to an ECG measuring device.

In a preferred embodiment the electrodes of the ergometry belt are assembled by means of a number of flexible strips that are pushed out by the electrode when the ergometry belt is worn and the flexible strips react to push back the electrode to the patient's body in order to tighten the electrode to the patient's body.

The principles and operation of the ergometry belt according to the present invention may be better understood with reference to the drawing and the accompanying description.

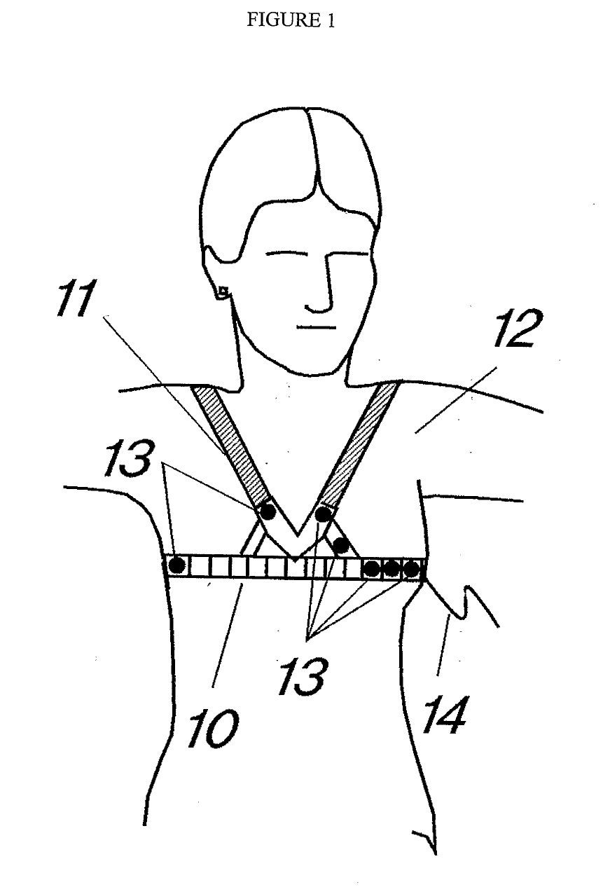

Referring now to the drawing, FIG. 1 illustrates the ergometry belt. The ergometry belt 10 is made of flexible and stretchable material 11—to be worn on a patient's body 12—with a plurality of electrodes 13 are disposed in designed places. Each electrode is connected via conductor (not shown) to a cable 14 that is connected to the ECG testing device.

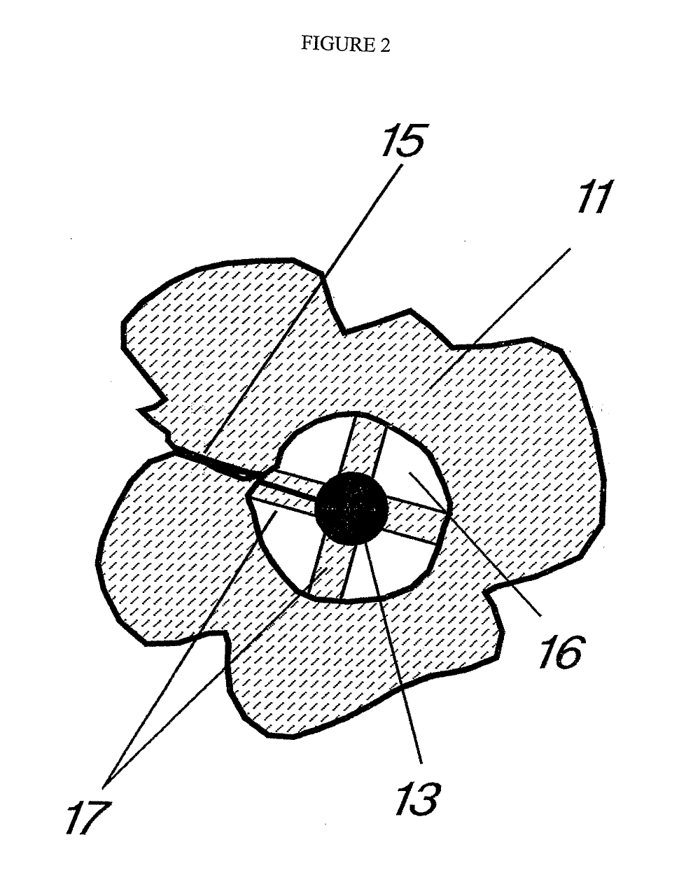

FIG. 2 illustrates the connection of an electrode to the belt. In the place—on the belt material 11—there is a hole 16 in the place where the electrode 13 is designed to be. The electrode 13 is located in the middle of the hole 16 and connected to the belt's material 11 by a number of flexible strips 17. The electrode 13 is connected through a conductor 15 that passes through one of the flexible strips.

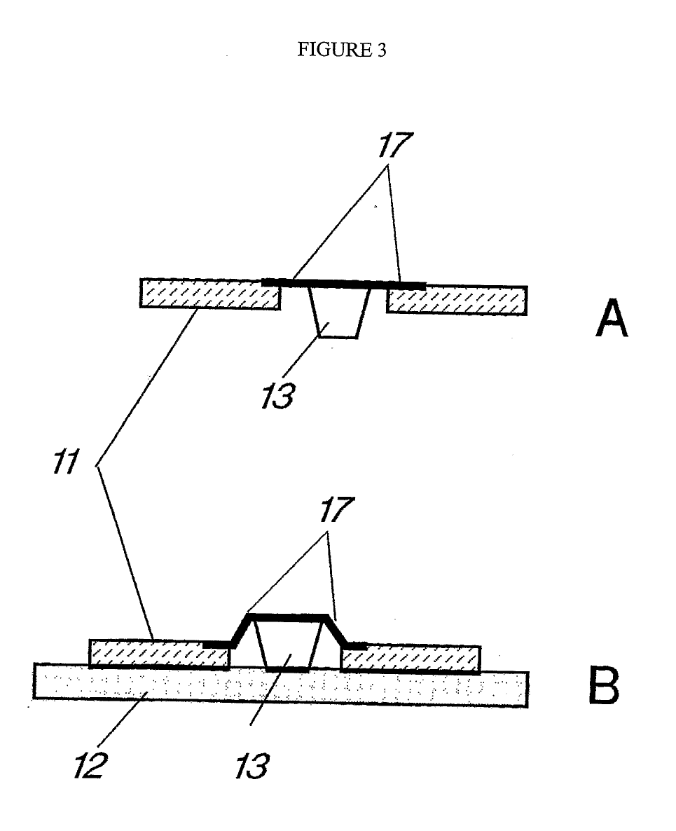

FIG. 3 illustrates a cross section of the electrode's connection area and way of the electrode tightening when the belt is worn. FIG. 3A illustrates the normal position of the electrode 13 that is connected by means of flexible strips 17 to the ergometry belt material 11. When the ergometry belt is worn—as is illustrated in FIG. 3B—the patient's body 12 pushes the electrode 13 up and the flexible strips 17 are stretched up. In a reaction, the flexible strips 17 push the electrode 13 and tighten it to the patient's body 12.

Although the invention has been described in conjunction with specific embodiments thereof, it is evident that many alternatives, modifications and variations will be apparent to those skilled in the art, accordingly, it is intended to embrace all such alternatives, modifications and variations that fall within the spirit and broad scope of the appended claims.

What is claimed is:

1. An ergometry belt particularly for use in ergometry (ECG stress test), comprising said ergometry belt made of a flexible and stretchable material, a plurality of electrodes disposed on the ergometry belt so that stretching of the belt will locate the electrodes in predetermined locations, a plurality of conductors adapted to be connected to a respective electrode, and a cable connected at one end thereof to each conductor and at another end thereof adapted to be connected to an ECG measuring device.

2. The ergometry belt of claim 1, wherein said conductors are located within the ergometry belt material.

3. The ergometry belt of claim 1, wherein said electrodes are placed on the patient's body at the correct anatomical places that are used to perform a ECG stress test, when said ergometry belt is worn.

4. The ergometry belt of claim 1, wherein instead of said plurality of electrodes a plurality of snaps, to which electrodes can be snapped, are disposed on the ergometry belt and wherein said ergometry belt further includes a disposal flexible thin layer with a plurality of electrodes that are disposed on said thin layer in accordance with the ergometry belt snaps' location, said flexible thin layer is attached to said ergometry belt in order to snap said electrodes to said ergometry belt in order to wear said ergometry belt with said attached flexible thin layer for performing an ergometry test.

5. The ergometry belt of claim l, wherein each of said electrodes is assembled to said ergometry belt by means of a number of flexible strips that are pushed out by said electrode when said ergometry belt is worn and said flexible strips react to push back said electrode to the patient's body in order to tighten said electrode to the patient's body.

6. The ergometry belt of claim 5, wherein a flexible membrane is used instead of the number of flexible strips.

7. The ergometry belt of claim 5, wherein said conductors are connected to said electrodes through one of said strips.
